(19) United States
(12) Patent Application Publication
(10) Pub. No.: US 2009/0275997 A1
(43) Pub. Date: Nov. 5, 2009

(54) VAGUS NERVE STIMULATION ELECTRODES AND METHODS OF USE

(76) Inventors: Michael Allen Faltys, Valencia, CA (US); James W. Broderick, Wellesley, MA (US); Kevin J. Tracey, Old Greenwich, CT (US)

Correspondence Address: SHAY GLENN LLP, 2755 CAMPUS DRIVE, SUITE 210, SAN MATEO, CA, 94403, US

(21) Appl. No.: 12/434,462

(22) Filed: May 1, 2009

Related U.S. Application Data
(60) Provisional application No. 61/049,740, filed on May 1, 2008.

(51) Int. Cl. A61N 1/36

(52) U.S. Cl. 607/2

(57) ABSTRACT

Described herein are systems, method and devices for modulating the cholinergic anti-inflammatory pathway. The systems described herein may include one or more implantable leads configured to be used to stimulate the inflammatory reflex. These leads typically include a flexible body region, a plurality of electrodes (or contacts) and may be used with a stylet or other inserter. The leads may also include one or more anchors. Exemplary leads may be intra-carotid sheath field-effect leads (“sheath FE” leads), carotid sheath cuff leads (“sheath cuff” leads), intracardiac leads, vagus nerve cuff leads (“vagus cuff” leads), and intravenous leads (“intravascular” leads). Leads (e.g., intravascular leads) may be chronic or acute.

### CROSS REFERENCE TO RELATED APPLICATIONS

This patent application claims priority to U.S. Provisional Patent Application Ser. No. 61/049,740 field May 1, 2008, titled “VAGUS NERVE STIMULATION ELECTRODES AND METHODS.”

This patent application may also be related U.S. Pat. No. 6,610,713, filed on May 15, 2001 and titled “INHIBITION OF INFLAMMATORY CYTOKINE PRODUCTION BY CHOLINERGIC AGONISTS AND VAGUS NERVE STIMULATION”; pending U.S. patent application Ser. No. 11/807,493, filed on Feb. 26, 2003 and titled “INHIBITION OF INFLAMMATORY CYTOKINE PRODUCTION BY STIMULATION OF BRAIN MUSCARINIC RECEPTORS”; pending U.S. patent application Ser. No. 10/446,625, with a priority date of May 15, 2001 and titled “INHIBITION OF INFLAMMATORY CYTOKINE PRODUCTION BY CHOLINERGIC AGONISTS AND VAGUE NERVE STIMULATION”; and pending U.S. patent application Ser. No. 11/318,075, filed on Dec. 22, 2005 and titled “TREATING INFLAMMATORY DISORDERS BY ELECTRICAL VAGUS NERVE STIMULATION.” This provisional patent application may also be related to pending U.S. Provisional Patent Application Ser. No. 60/968,292, titled “DEVICES AND METHODS FOR INHIBITING GRANULOCYTE ACTIVATION BY NEURAL STIMULATION”, and Ser. No. 60/982,681, titled “TRANSCUTANEOUS VAGUS NERVE STIMULATION REDUCES SERUM HIGH MOBILITY GROUP BOX 1 LEVELS AND IMPROVES SURVIVAL IN MURINE SEPSIS”. Each of these patents and patent applications are herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

The devices, systems and methods described herein relate generally to the modulation of inflammation, and particularly to the modulation of the cholinergic anti-inflammatory pathway by electrical stimulation.

### BACKGROUND OF THE INVENTION

Inflammation is a complex biological response to pathogens, cell damage, and/or biological irritants. Inflammation may help an organism remove injurious stimuli, and initiate the healing process for the tissue, and is normally tightly regulated by the body. However, inappropriate or unchecked inflammation can also lead to a variety of disease states, including diseases such as hay fever, atherosclerosis, arthritis (rheumatoid, bursitis, gouty arthritis, polymyalgia rheumatic, etc.), asthma, autoimmune diseases, chronic inflammation, chronic prostatitis, glomerulonephritis, nephritis, inflammatory bowel diseases, pelvic inflammatory disease, reperfusion injury, transplant rejection, vasculitis, myocarditis, colitis, etc. In autoimmune diseases, for example, the immune system inappropriately triggers an inflammatory response, causing damage to its own tissues.

Inflammation can be classified as either acute or chronic. Acute inflammation is the initial response of the body to harmful stimuli and is achieved by the increased movement of plasma and leukocytes from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterized by simultaneous destruction and healing of the tissue from the inflammatory process.

The nervous system, and particularly the vagus nerve, has been implicated as a modulator of inflammatory response. The vagus nerve is part of an inflammatory reflex, which also includes the splenic nerve, the hepatic nerve and the trigeminal nerve. The efferent arm of the inflammatory reflex may be referred to as the cholinergic anti-inflammatory pathway. For example, Tracey et. al., have previously reported that the nervous system regulates systemic inflammation through a vagus nerve pathway. This pathway may involve the regulation of inflammatory cytokines and/or activation of granulocytes. Thus, it is believed that appropriate modulation of the vagus nerve may help regulate inflammation.

A system for stimulating one or more nerves of the inflammatory reflex may include one or more electrical leads which may be implanted acutely or chronically, and may be positioned adjacent or in contact with the vagus nerve or other nerves of the inflammatory reflex, and particularly the cholinergic anti-inflammatory reflex.

Currently available systems for stimulating nerves of the inflammatory reflex such as the vagus nerve are generally not appropriate for stimulation of the vagus nerve to regulate inflammation. The configuration of the electrodes and stimulators, including the configuration of the stimulating electrodes of the electrical leads, in conjunction with the level, duration and frequency of stimulation, are critical to inhibiting or modulation of the inflammatory response appropriately (e.g., without desensitizing the inflammatory reflex).

For example, US Patent Application publication numbers 2006/0287678, 2005/0075702, and 2005/0075701 to Shafer describe a device and method of stimulating neurons of the sympathetic nervous system, including the splenic nerve, to attenuate an immune response. Similarly, US Patent Application publication numbers 2006/0206155 and 2006/010668 describe stimulation of the vagus nerve by an implanted electrode. US Patent Application publication number 2007/0027499 describes a device and method for treating mood disorders using electrical stimulation. US Patent Application publication number 2006/0229677 to Moffitt et al. describes transvascularly stimulating a nerve trunk through a blood vessel. U.S. Pat. No. 7,269,457 to Shafer et al. also describes a system for vagal nerve stimulation with multi-site cardiac pacing. All of these published patent applications and issued patents describe systems and methods for stimulating nerves, including the vagus nerve. However, none of these publications teach or suggest stimulating the inflammatory reflex, including the vagus nerve, using a system or method that would prevent desensitization of the inflammatory reflex.

Electrical leads and systems appropriate for stimulating the inflammatory reflex (e.g., the vagus nerve) must be configured so as to prevent desensitization of the inflammatory reflex as well as to allow stimulation at a strength, duration and frequency that will effectively modulate the inflammatory reflex. Modulation of the inflammatory reflex is possible only within a controlled range of signal strengths, durations and frequency; stimulation outside of this range will result in either under-driving (e.g., failing to modulate the inflammatory reflex) or over-driving (desensitizing the inflammatory reflex). The electrodes and systems described above, including 2006/0287678, 2005/0075702, and 2005/0075701 to Shafer, include electrodes and systems that are not attuned to the inflammatory reflex. For example, the cuff electrodes and nerve-contacting electrodes such as those described in the 2006/0287678 applications will likely contact the never, and rapidly desensitize the effect on the inflammatory reflex. Recent preliminary data suggests that the inflammatory reflex may be triggered by mechanical manipulation of the nerve (e.g., by contacting the vagus nerve with an electrode). Thus cuff electrodes or electrodes implanted to contact the nerve directly may desensitize the modulation of the inflammatory reflex.

In addition, the response to the inflammatory reflex must be within an appropriate range of stimulation values and parameters, including intensity (or strength) of the stimulation seen by the nerve, bursting stimulation parameters (e.g., duration and in-burst frequency, as well as the frequency between bursts of stimulation), and location of stimulation on the inflammatory reflex. Outside of the prescribed ranges of stimulation seen at a nerve of the inflammatory reflex, the resulting stimulation may not be effective, and may inhibit correct stimulation (e.g., by desensitization).

Thus, there is a need for electrical leads and systems that include electrical leads are configured to appropriately modulate the inflammatory reflex without desensitizing the response.

### SUMMARY OF THE INVENTION

Described herein are methods and systems for modulating the inflammatory reflex, by stimulating the Cholinergic Anti-inflammatory Pathway (CAP). In particular, described herein are systems including one or more leads that may be used to modulate the inflammatory reflex. These leads are implantable or insertable into a subject's body and are configured to allow controlled stimulation of the subject's cholinergic anti-inflammatory pathway (e.g., at least a portion of the subject's vagus nerve). Exemplary leads may be configured as: intra-carotid sheath field-effect leads (“sheath FE” leads), carotid sheath cuff leads (“sheath cuff” leads), intracardiac leads, vagus nerve cuff leads (“vagus cuff” leads), and intravenous leads (“intravascular” leads). Leads may be chronic or acute. In general, the leads described herein may include a flexible body, a plurality of electrodes, and a stylet.

The Cholinergic Anti-inflammatory Pathway (CAP) is a neural pathway in which the efferent vagus nerve regulates systemic cytokine levels through a nicotinic acetylcholine receptor containing the α7 subunit. The response, which attenuates cytokine levels, can be achieved through direct or indirect activation of the corresponding efferent vagus fibers leading the spleen. The CAP can be elicited via afferent or effect pathways using different physical actuator or electrode approaches and locations, and corresponding specific stimulus parameters. Specifically actuators or electrodes are arranged physically to activate the vagal or afferent or efferent fibers or the splenic nerve efferent fibers. These approaches and parameters are designed to: avoid undesirable side effects such as intestinal motility and cardiac effects; elicit a controlled dynamic dose effect as dictated by the system response and pharmacokinetics of anti-inflammatory and pro-inflammatory cytokines; co-modulate both anti-inflammatory and pro-inflammatory cytokines; and not induce a tachyphilaxis.

Reflex responses diminish in time due to repeated stimulation, an effect termed tachyphilaxis. Tachyphilaxis is avoided by not over stimulating (e.g., overdriving) the CAP. This includes, but is not limited to, stimulating only a brief period every hour or every day of between 10 seconds and 5 minutes, delivering an impulse function to the system (2-10×) threshold for 1-60 seconds between every week and every month, alternating between levels and frequencies every other month (5 Hz at 200% threshold, 20 Hz at 100% of threshold). Tachyphilaxis may be triggered by leads or electrodes that contact the nerve (e.g., nerve cuffs), if they are not appropriately designed, since stimulation of the nerve at the very low levels sufficient to modulate the CAP have been evidenced. Thus the configuration of the electrode or lead, as well as the duration and frequency of bursts should be configured to avoid this effect. This includes surgical cuffs and movement isolation buffers to prevent body movement from being transfers to the lead and the nerve.

The CAP includes cytokine receptors that are relayed to the brainstem via afferent vagal fibers. The brain activates the vagal cholinergic efferent fibers that terminate in the celiac-superior mesenteric plexus ganglia that then relay to the catecholaminergic nerve fibers that innervate the spleen. In the spleen, those fibers are believed to modulate α7 surface receptors on macrophages, affecting cytokine production of the individual cells and the system. This effect persists for days and may be linked to the lifespan of macrophages (a few days) or a compounded system response. Due to the high profusion rate of macrophages through the spleen, short durations of stimulation reaching the spleen may have long lasting and profound effects on cytokine production. Described herein are several embodiments of neural actuators and leads and associated stimulation parameters to achieve modulation of the inflammatory pathway (e.g., CAP), in a controlled and desired fashion.

In general, electrical activation of the CAP may be performed by any of several electrode placements. Examples of these are listed below. The endpoint of all of the different configurations is cytokine modulation, presumably through the spleen. In the list below, efferent methods travel directly to the spleen, while afferent methods may target the reflex mechanisms in the brain which in turn triggers the spleen.

We contemplate the following configurations of electrodes and systems for modulation of the CAP: (1) Multipolar cutaneous pinna electrode to actuate the afferent vagus to activate the efferent to the spleen; (2) Multipolar right cervical vagus cuff to actuate afferents or efferents to the spleen; (3) Multipolar left cervical vagus cuff to actuate afferents and then right vagal efferents to the spleen; (4) Multipolar right cervical subcutaneous field effect electrode to actuate afferents or efferents to the spleen; (5) Multipolar left cervical field effect electrode to actuate afferents and then right vagal efferents to the spleen; (6) Intravascular multipolar right cervical field effect electrode to actuate afferents or efferents to the spleen; (7) Intravascular multipolar left cervical field effect electrode to actuate afferents and then right vagal efferents to the spleen; (8) Multipolar subclavian vein (SVC) to activate the vagus afferents or efferents; (9) Multipolar splenic cuff to activate the splenic efferents; and (10) Multipolar splenic vein or artery intravascular electrode to actuate the splenic efferents.

In particular, described herein are methods of modulating inflammation by stimulation of the cholinergic anti-inflammatory pathway (CAP). Methods may include the implantation of one or more leads (each lead containing one or more electrodes) within a region adjacent to, but not touching, the vagus nerve, or another nerve of the anti-inflammatory reflex. As mentioned, contacting the vagus nerve may result in inadvertent activation of the inflammatory reflex and lead to de-sensitization. For example, described herein are methods of modulating inflammation by stimulation of the cholinergic anti-inflammatory pathway including the steps of: positioning a flexible lead within a blood vessel; confirming that each of a plurality of electrodes on the flexible lead are secured against the wall of the blood vessel; anchoring the flexible lead within the blood vessel; and modulating the cholinergic anti-inflammatory pathway by applying energy to one or more stimulation electrodes on the flexible lead to stimulate the vagus nerve.

As used herein a “blood vessel” is intended to include any blood vessel of the body (e.g., the human body), including veins and arteries, and particularly those veins, arteries and other blood-passing organs that are proximally adjacent to a part of the inflammatory reflex (e.g., the vagus nerve). Thus, the step of positioning the lead within the blood vessel may comprise positioning the lead within the internal jugular vein (e.g., by inserting the lead using a subclavian approach), the coronary sinus, or the pulmonary artery.

In some variations, the method may also include the step of determining which electrodes on the lead are best used as the stimulation electrodes. For example, different electrodes (or pairs of electrodes) on the lead may be used to apply energy to alter the subject's heart rate (e.g., inducing mild bradycardia). Thus, by cycling through stimulation from different electrodes on the lead and monitoring the heart rate (to detect the effect of stimulation of the vagus nerve on the heart rate), the electrode or pair of electrodes that is most effective for stimulating the vagus nerve can be determined, corresponding to the “stimulation electrode(s)” on the lead.

In general, the flexible lead used for inserting into a blood vessel may be configured so that it does not substantially occlude the vessel, or interfere with the passage of blood within the vessel. Thus, the lead may include a passageway through the body of the lead for passing blood. In some variations, the lead is configured to lie against the walls of the blood vessel. For example, the lead may be a helical, substantially flat lead that wraps around the wall of the vessel. Examples of these leads are provided below. The lead may be biased so that is applies a preset (e.g., bias) force to self-expand against the wall of the vessel.

The method may also include the step of confirming that each of the electrodes on the flexible lead are secured against the wall of the blood vessel. In this variation, the electrodes are oriented against the wall of the vessel (aimed “outward”) and the back of the lead (and electrodes) that faces within the vessel is insulated, to prevent loss of current. The connection between the wall of the vessel and the electrodes can be confirmed by testing the impedance of each electrode. A low impedance may indicate that blood from the lumen of the vessel is contacting the electrode surface. In some variations the electrodes may be further ‘pushed’ against the wall of the vessel by further expanding the lead. In some variations, when a low-impedance electrode is detected, it may be removed from the pool of potential stimulation electrodes.

As mentioned, the leads may be used within the patient either acutely or chronically. Thus, a lead may be implanted into the patient for long-term use, or it may be implanted for removal. A controller (e.g., within a housing) may also be implanted into the patient. The controller is typically configured to control the application of energy from the stimulation electrodes. Thus, the controller may include or be connected to a power supply such as a pulse generator, etc.

In general, the power applied to the stimulation electrodes is sufficient to modulate the cholinergic anti-inflammatory pathway, for example, by stimulation of the vagus nerve. The power applied may be sufficient to modulate inflammation via the CAP without desensitizing the CAP. For example, the power applied may be a short-period of stimulation (e.g., less than five minutes, less than 1 minute, less than 30 seconds, less than 10 seconds, less than 5 seconds, less than 1 second, etc.) followed by a long “off-period” during which stimulation is not applied. The off-period may be greater than 30 minutes, greater than 1 hour, greater than 2 hours, greater than 4 hours, greater than 8 hours, greater than 12 hours, greater than 24 hours, greater than 36 hours, greater than 48 hours, etc.). The short-period of stimulation may comprise a burst of high-frequency, relatively low-power stimulation. For example, the stimulation may be applied as a burst of 10 Hz-1 Gz pulses of less than 5 V, less than 1 V, less than 0.1 V, less than 0.01V, etc. In general, the energy applied is substantially lower than the energy that would be applied from the same electrode to modify the subject's heart rate. For example, the energy applied may be a fraction of the energy required (e.g., threshold energy) to modify heart rate from the same stimulation electrode, such as 80%, 75%, 70%, 60%, 50%, 40%, 30%, 20%, 10%, 5%, 1%, or less than 1%.

In some variations, the optimum energy applied to modulate the anti-inflammatory pathway may be customized to a patient or class of patients (e.g., patients within a certain age, weight, height, gender, etc.). Thus, the optimum energy may be customized by periodically measuring markers for inflammation (as described in many of the references incorporated by reference above) after stimulating at a variety of power levels and frequencies.

Other, similar variations include the use of a carotid sheath lead. For example, a method of modulating inflammation by stimulation of the cholinergic anti-inflammatory pathway may include the steps of: positioning a flexible lead in a carotid sheath so that the lead does not contact the vagus nerve; anchoring the lead within the carotid sheath; and modulating the cholinergic anti-inflammatory pathway by applying energy to one or more stimulation electrodes on the flexible lead to stimulate the vagus nerve.

The method may also include the step of determining the stimulation electrode or electrodes from among a plurality of electrodes on the flexible lead by applying energy from among the electrodes on the flexible lead and monitoring heart rate, as described above. In some variations, the step of positioning the flexible lead in the carotid sheath comprises inserting the lead using a subclavian approach.

The method may also include the step of coupling the flexible lead to a stylet to aid insertion before inserting the flexible lead into the carotid sheath. The stylet may be removed from the flexible lead after insertion.

Also described herein are methods of modulating inflammation by stimulation of the cholinergic anti-inflammatory pathway using a carotid sheath cuff lead. For example, the method may include the steps of: positioning a carotid sheath cuff lead around a carotid sheath so that the carotid sheath cuff lead does not contact the vagus nerve; anchoring the carotid sheath cuff lead around the carotid sheath so that stimulation electrodes located within the carotid sheath cuff lead are oriented towards the vagus nerve within the carotid sheath; and modulating the cholinergic anti-inflammatory pathway by applying energy to one or more stimulation electrodes within the carotid sheath cuff lead to stimulate the vagus nerve.

The step of positioning the carotid sheath cuff lead around the carotid sheath may performed by surgically cutting down to the carotid sheath. In some variations, the carotid sheath cuff is sutured around the carotid sheath. The carotid sheath cuff may be insulated on the outer surface of the cuff to prevent stimulation of surrounding tissue.

### BRIEF DESCRIPTION OF THE DRAWINGS

FIG. 1A is a cross-section though an artery including an intravascular lead. FIG. 1B shows a partial side view of the artery and lead shown in FIG. 1A.

FIGS. 2A-2F illustrate another variation of a flexible intravascular lead having a plurality of electrodes. FIGS. 2A and 2B show top and side views, respectively. FIG. 2C is an enlarged view of one electrode region. FIG. 2D shows a side perspective view of the portion of the lead shown in FIG. 2C, with the flexible body portion removed. FIG. 2E illustrates a partial cut-away view of one variation of the lead of FIG. 2A within a blood vessel. FIG. 2F shows an overmolding mandrel that may be used to form the lead shown in FIG. 2A.

FIGS. 3A-3C illustrate another variation of a flexible intravascular lead having a plurality of electrodes. FIG. 3A is a top view of one variation of the lead, while FIGS. 2B and 2C illustrate side views of top and bottom (“inside” and “outside” layers forming the lead.

FIGS. 4A and 4B show front and side views, respectively, of one variation of a delivery device for an intravascular lead.

FIG. 5A shows one variation of an intra-carotid sheath electrode lead. FIGS. 5B and 5C illustrate the sheath electrode lead of FIG. 5A implanted into a subject's carotid sheath.

FIG. 6A shows one variation of a carotid sheath cuff electrode lead, and FIGS. 6B and 6C illustrate the cuff electrode lead of FIG. 6A implanted around a subject's carotid sheath.

FIG. 7A shows one variation of an intracardiac electrode lead, and FIGS. 7B and 7C illustrate the intracardiac electrode lead of FIG. 7A within the coronary sinus.

FIG. 8A shows a sheath electrode lead, and FIGS. 8B and 8C illustrate the sheath electrode lead around the vagus nerve.

FIG. 9A shows another variation of an intravascular electrode lead, and FIGS. 9B and 9C illustrate the intravascular electrode lead implanted in a subject's internal jugular vein.

FIG. 10 is a grid illustrating different variations of field effect electrode architectures.

FIGS. 11A-11C illustrate different stylet variations that may be used with some of the lead configurations described herein. FIGS. 11D and 11E illustrate cross-sections through different lead configurations.

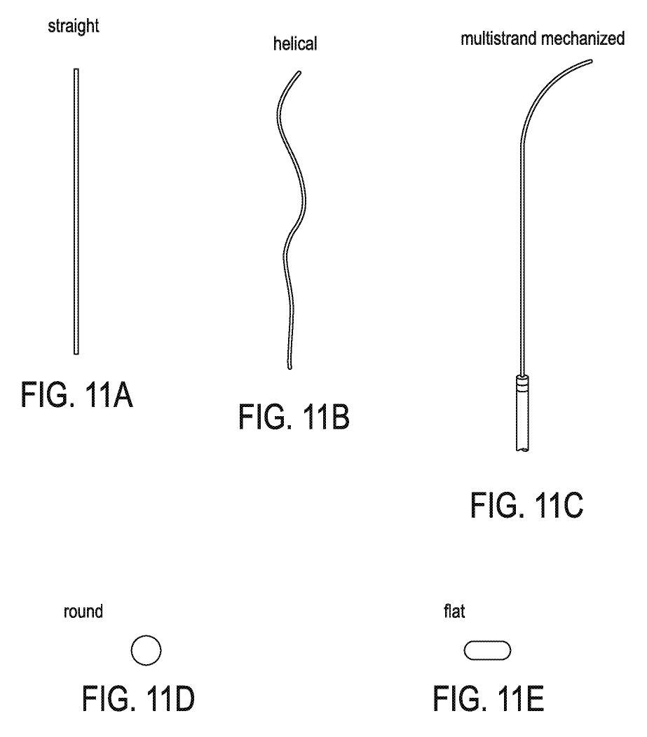

### DETAILED DESCRIPTION OF THE INVENTION

The stimulator configurations and methods of inserting them, as well as systems including them are intended for use in modulating the inflammatory reflex, which may also be described as modulating the Cholinergic Anti-inflammatory Pathway (CAP). A stimulator may include a lead (e.g., an electrical lead) and a controller for regulating the energy applied to the subject by the lead. In some variations the stimulator may also include a power source or power supply providing power to the controller, and for acting as the source of energy applied by the lead. Since, in general, the energy applied to the CAP to modulate the inflammatory reflex is much lower than the energy applied to modulate other vagus effects (such as heart rate, blood pressure, intestinal response, etc.), the source of energy and/or the controller may be substantially smaller and more compact than existing vagus nerve stimulators, for example.

In general, the systems (e.g., a stimulator) described herein may include one or more leads. A lead may include a flexible body, a plurality of electrodes, and (in some variations) a stylet. The flexible body may be tubular and/or hollow, and may have a round, oval, or flat cross-section. The flexible body is typically made of a biocompatible material and may be configured to be inserted or implanted into a subject. For example, the flexible body may be formed of a polymeric material. In some variations, the material forming at least a portion of the flexible body is an electrically insulative material. The electrodes of the lead may be attached to the flexible body, or they may be embedded within the flexible body. For example, the electrodes may be exposed through windows or openings in the body.

The flexible body may also include one or more longitudinal passageways. For example, the lead may include a stylet channel through the body of the lead into which a stylet may be passed. Thus, in some variations, the stylet is removable from the rest of the lead. In some variations, different stylets (e.g., stylets having different shapes, sizes, stiffness, or other properties or combination of properties) may be used in the same lead by exchanging them in the passageway.

The flexible body is typically elongate, and may have a uniform or non-uniform cross-sectional thickness. For example, the flexible body may be tapered. In some variations the flexible body includes a cuff or cuff region. For example, the flexible body may be C-shaped or O-shaped, and my include electrodes on the inner surface.

The leads typically include a plurality of electrodes. Electrodes may be multipolar, bipolar, or monopolar electrodes. In general, the leads include multipolar field-effect electrodes. The electrodes typically include one or more electrical contact surfaces which are electrically connected to a connector (e.g., wire, channel, etc.) projecting proximally down the length of the lead for connection to the controller and/or electrical stimulator. Leads which are not hardwired to connect to a controller (or that are wirelessly connected to the controller) are also possible. Electrodes may be discrete electrodes that are individually connected to a longitudinal connector, or multiple electrodes may be connected to the same longitudinal connector. The electrodes may be spaced from each other in any appropriate fashion.

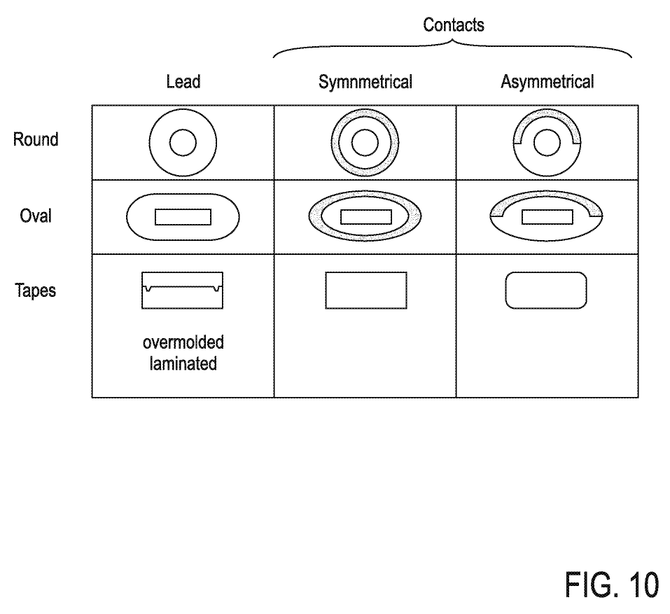

Electrodes on the lead may have any appropriate surface geometry. For example, the electrodes may be ring electrodes (e.g., circumferentially spanning the body of the lead), or may span only a portion of the circumference of the electrode. The grid shown in FIG. 10 illustrates some variations of the electrodes and lead bodies described above. For example, in FIG. 10, the cross-section of the lead in the region of the electrodes is shown as round, oval or tapered (substantially flat). In this example, the body region includes a hollow opening, as described above, into which the stylet may pass. The opening may be round or non-round, and may be keyed to the stylet. The figure on page 6 of Appendix A also illustrates ring electrodes (symmetrical electrodes) and partial (asymmetrical) electrodes.

The stylet may be used to help implant the leads described herein, since they may be used to impart rigidity, stiffness and in some instances steerability. A stylet for use in any of the leads described herein is typically a relatively stiff, elongate structure. In some variations the stylet includes a proximal handle region. The stylet may be steerable. For example, the stylet may be formed of a plurality of wires that may be selectively tensioned to steer, bend, or curve the device. In some variations the style is pre-bent or pre-formed so that it assumes a curved (or curing) shape. The stylet may have any appropriate cross-section, including (but not limited to) round, oval, and flat. The figures shown in FIGS. 12A-12E illustrate different stylet variations as described herein.

As mentioned, any of the lead variations described herein may be used as part of a system or as a component of a device, including a vagus nerve stimulator or a system for modulating the inflammatory reflex. Any of these leads may also be used with one or more anchors or retainers for retaining the lead in position within the subject's body. In some variations the leads are self-anchoring or are configured to expand and interact with the tissue so that they may be secured in place.

Examples of the different types of leads, as well as methods of using them, are briefly described below. Although each of these lead types (e.g., intra-carotid sheath field-effect leads, carotid sheath cuff leads, intracardiac leads, vagus nerve cuff leads, and intravenous leads) is described separately, features of any of these leads may be used with any of the other leads. Methods of using and manufacturing these leads are also provided, and may be adapted for any of the other lead types illustrated. Any of these leads (e.g., intravascular leads) may be chronic or acute leads.

The stimulation protocol (and thus the controller, stimulator, power supply, etc.) used for each of these different leads may be matched to the location that the lead (or stimulator including the lead) is to be inserted into. Part two, below, illustrates systems and variations of systems for implantation in different body regions. In addition, the stimulation protocol for use with each system (including the lead and/or stimulator) may also be configured based on the type of lead used. For example, Table 1, below illustrates exemplary ranges and stimulation protocols for each of the different types of leads indicated. These ranges are exemplary.

TABLE 1
Exemplary Stimulation parameters

| | | | Stimulus |
|---|---|---|---|
| | | Stimulus | current |
| | | current | threshold of |
| | Fiber | threshold | side effects |
| Electrode type | Type | of CAP (T) | (MAX) |
| Cutaneous Pinna | Afferent | 100 uA-2 mA | 2 mA-10 mA |
| Right Cervical Vagus | Afferent | 100 uA-2 mA | 1-10 mA |
| Cuff | | | |
| Right Cervical Vagus | Efferent | 200 uA-4 mA | 1-10 mA |
| Cuff | | | |
| Right Cervical Field | Afferent | 200 uA-4 mA | 2-20 mA |
| Effect | | | |
| Right Cervical Field | Efferent | 400 uA-8 mA | 2-20 mA |
| Effect | | | |
| Intravascular Right IJV | Afferent | 400 uA-4 mA | 2-20 mA |
| Intravascular Right IJV | Efferent | 800 uA-8 mA | 2-20 mA |
| Intravascular SVC | Afferent | 200 uA-2 mA | 2-20 mA |
| Intravascular SVC | Efferent | 800 uA-8 mA | 2-20 mA |
| Splenic Cuff | Efferent | 200 uA-4 mA | 2-20 mA |
| Splenic Intravascular | Efferent | 1-10 mA @ 20 Hz | 2-20 mA |

The duration of stimulation that is transmitted to the splenic nerve (and likely the spleen) is believed to modify macrophages in the spleen so that their response characteristics to infection. The duration (intensity) of the stimulation may be expressed in seconds for a specific pulse width (50-500 uS). In addition, the stimulation may be repeated after a period of off-time, or non-stimulation at those parameters. The period of off-time may extend from minutes, to hours, to days (e.g., 2 hours, 4 hours, 8 hours, 12 hours, 24 hours, 48 hours, etc.).

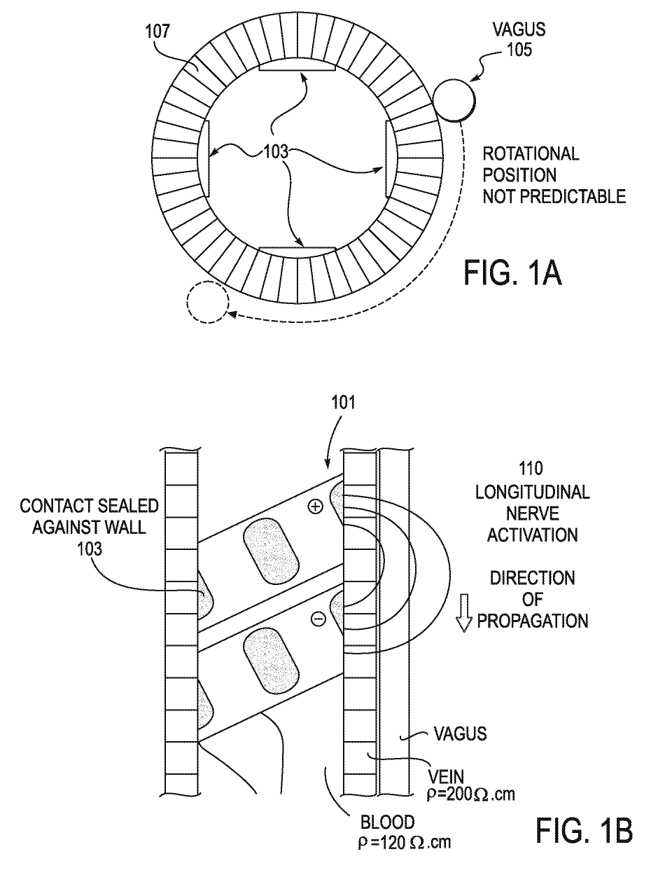

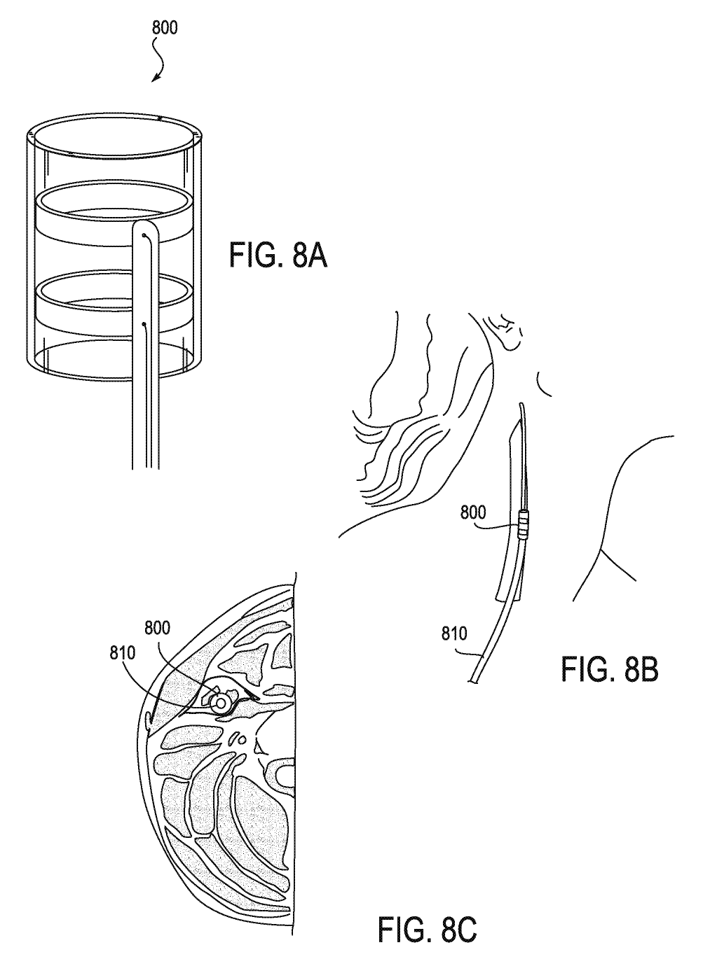

Examples of different configurations, and systems including them, as well as method of inserting and using them, are provided below. In some variations, the devices include intravascular leads that are configured to be used within a subject's vasculature, including the heart. Intravascular leads (which include “intravenous” leads) may be configured as field-effect (e.g., multipolar) leads that may be placed in a body lumen, such an artery, vein, sinus, or the like. FIGS. 1A-5C illustrate variations of this type of lead. As mentioned above, an intracardiac lead may also be one type of intravascular, lead (as shown in FIG. 8A-8C and 10A-10C). In some variations (e.g., FIG. 10A-10C), an intravascular lead is used in the internal jugular vein (IJV). Other illustrations of intravascular leads are given below, and shown in the figures.

FIGS. 1A and 1B show one variation of an intravascular lead 101 that may be used. The lead may operate from within the pulmonary artery 107 or coronary sinus, with contacts 103 on the outside of the electrode body (e.g., pointing outwards), rather than on the inside, as in the standard cuff configuration in which the electrodes are oriented towards the inside. The external (outward-facing) contacts 103 typically press up against the wall of the artery 107 or sinus and almost all of the current is transmitted through the walls between bipolar contacts 110. A pair of bipolar contacts 103 may be chosen to track in close proximity to the vagus nerve, as indicated in FIG. 1B.

The proximity of the vagus nerve may be evaluated objectively and interoperatively by inducing mild bradycardia. For example, during implantation of the lead, after seating the lead against the wall of the vessel or chamber, leads (or pairs of leads) may be stimulated to trigger bradycardia. Leads (including adjacent pairs of leads) may be stimulated sequentially to determine if result in stimulation of the vagus nerve and therefore bradycardia. The stimulation applied to trigger bradycardia is typically much greater than the stimulation applied to modulate the inflammatory reflex. Multiple cycles of stimulation (e.g., increasing the applied power with each cycle) may be performed until bradycardia (or other confirmed vagus stimulation) is detected. Other method of confirmed vagus nerve stimulation include direct measurement of stimulation of the vagus (e.g., by sensing electrode) or by other determinations of vagal tone. When induced with sufficiently low stimulation thresholds, the electrode can be assured to be in the proper location.

In the example shown in FIGS. 1A and 1B, eight electrodes 103 may allow 90° of spatial selectivity with bipolar pairs arranged longitudinally. The number of electrodes can be increased or decreased and can be selected based upon the desired selectivity of the electrode. The electrodes may be sealed against the walls of the sinus or artery to direct current through the walls and prevent the low impedance blood from shunting the current. The insulating electrode substrate may prevent current flowing to the blood through the back of the electrode.

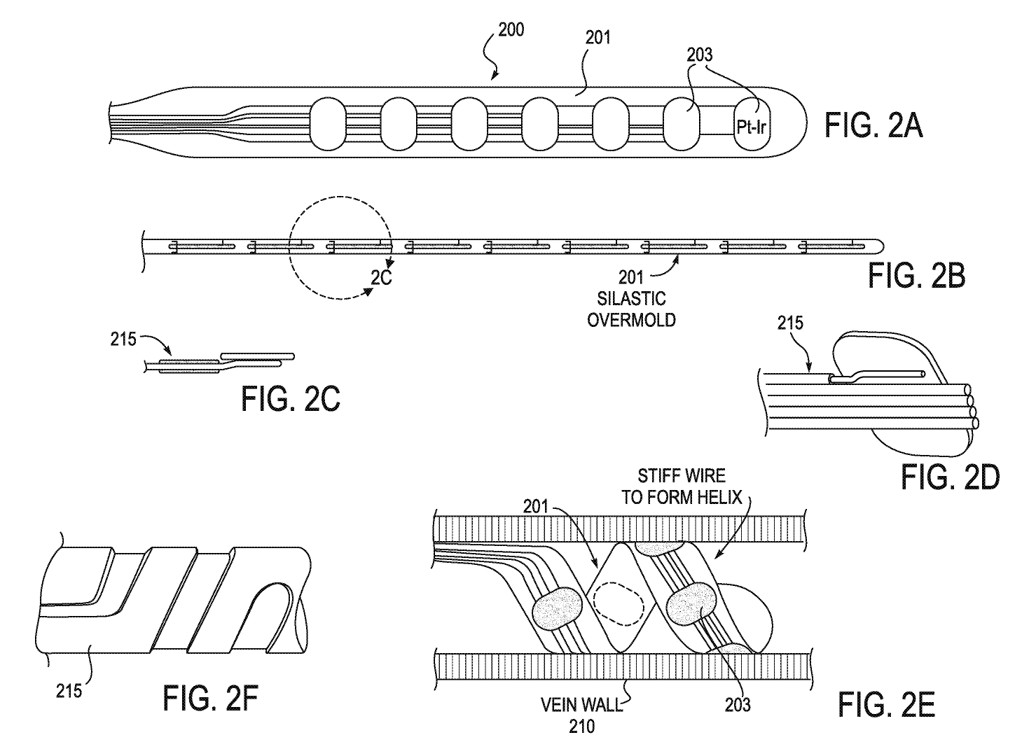

In another exemplary embodiment, illustrated in FIGS. 2A-2F, the lead body 201 is made of a flexible polymer (e.g., a silastic material in this example), and the body is embedded with electrically conductive (e.g., conductive metal) materials to form the plurality of electrodes 203 that are robust and compliant. The stiffness may be controlled by the wire characteristics (connecting the electrodes) and the hardness (durometer) of the silastic. In some variations the conductive material is a conductive polymer, which may enhance flexibility.

The lead may be constructed by aligning insulated wires in parallel. For example, in FIG. 2A-2F, Pt—Ir plates are welded to selected individual wires through the insulation. This is shown in more detail in FIGS. 2C and 2D, which show enlarged views of one electrode 203 of the lead 200.

A lead such as the one shown in FIG. 2A-2F may be fabricated by molding. In some variations, the lead is formed by connecting the electrode surfaces to one or more conductive regions. For example, a flexible but supporting scaffolding may be formed, and the electrodes connected to them before adding the flexible backing. For example, the most apical contact may be welded to one or more wires 211 to maintain its apical stiffness and robustness. This preform can then be wrapped around a mandrel and tempered (which will burn off insulation) to hold shape. The contacts are spaced in the pre-form to conform to a specified angular spacing for a particular mandrel. A tight fitting tube is fitted over the mandrel 215 and the silastic is injection molded. Each wire coming off the electrode is thus helically wound to withstand the constant movement in the neck.

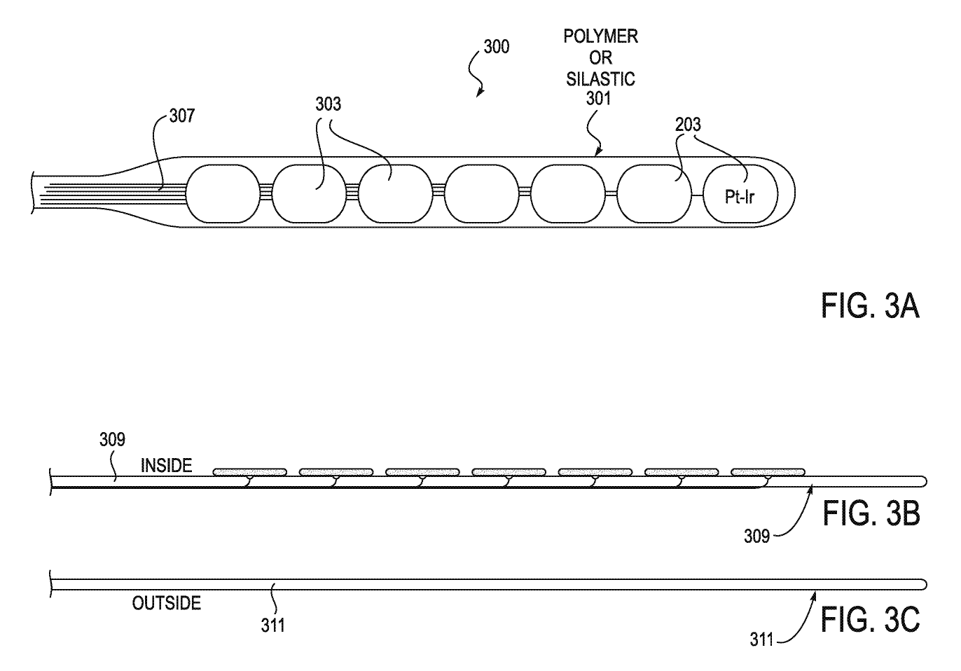

FIG. 3A-3C illustrate another variation of an intravascular lead 300 that gets its shape and stiffness not from the wire members, but the backing/support material 301. In this example, the backing material is formed from a lamination of two materials. Two sheets 309, 311 of silastic or polymer are used to construct the electrode. The contacts 303 are embedded in the first sheet 309 with the electrode wires 307 coming out the other side. These contacts 303 and wires 307 can be constructed using plates and wires or lithographic techniques. These two sheets are then bonded together around a mandrel. The diameter of the mandrel may be slightly larger than the diameter of the vessel into which the electrode will be implanted, allowing the lead to be pre-biased in an expanded configuration. The backing sheet 301 in this example has two functions. First, it may hold the shape of the mandrel and thus will hold the electrode against the inside walls of the vessel (e.g., artery or sinus) when the vessel has a smaller diameter than the mandrel. It may also form an insulating and/or protective shield for the individual wires connecting to the contact pads.

Before implantation into the body, the intravascular lead may be sized to fit the implantation site. Sizing can be performed by one of skill in the art (e.g., an interventional cardiologists) utilizing various accepted techniques. An impedance catheter can also be used to measure the volume indirectly through volume conductance if the other techniques are not desirable.

In some variations, the lead is deployed from a delivery device that includes a rod or stylet. The rod or stylet provides an elongated, typically flexible but supportive structure that will hold or constrain the lead in a delivery configuration until it is deployed into a deployed configuration. For example, the lead may be delivered and deployed by wrapping it tightly against a stylet or catheter and then releasing the assembly when it is approximately positioned. In another variation, the lead includes an inner (e.g. central) passage or channel through which the stylet may pass. When inserted, the stylet may hold the lead in the delivery configuration (e.g., tightly wound), and removal of the stylet may allow it to expand into the vessel.

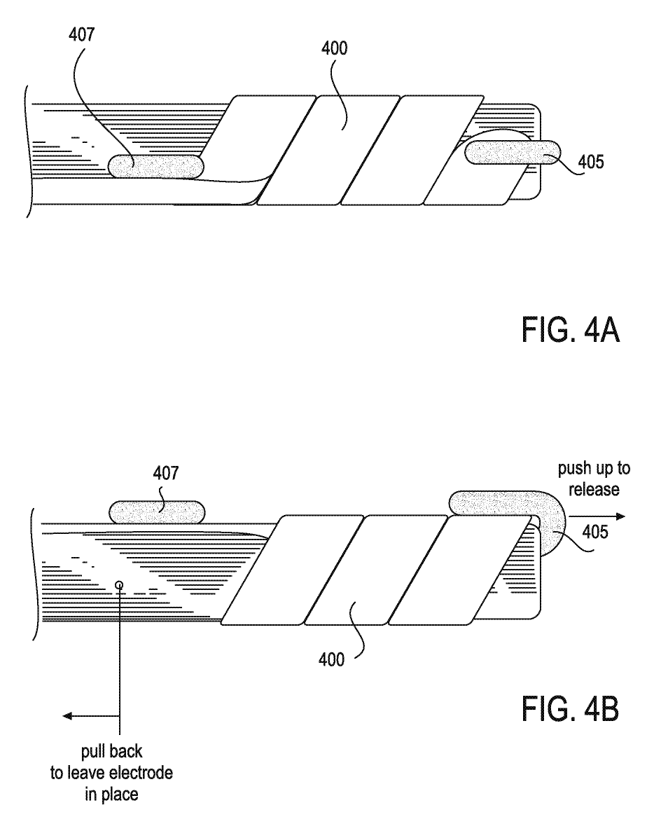

FIGS. 4A and 4B illustrate another variation of a delivery device, in which the lead is wrapped tightly around a delivery catheter or stylet that includes two or more retainers for holding it wrapped around the delivery device. In FIG. 4A, the inner core retainer pins 405, 407 may be removed (by pulling pack or outward) to release the lead for deployment. In some variations the lead is configured to be used acutely, and is removable. Thus, the lead may include a tether or other structure allowing later retraction or removal. For example, a retainer (e.g., pin) may be permanently attached to one end of the lead, allowing it to be reversibly expanded. For example, a core pin may be extended and rotated to expand the lead, and then rotated in the opposite direction to retract when stimulation is complete.

In any of these variations, once a lead is in place, impedances can be measured to assure that the contacts are against the walls of the body lumen. If an electrode does not fit the walls tight enough then the impedances may vary greatly across contact, as some electrodes will be in contact with the low impedance blood and some will be against the higher impedance walls.

After the impedance test is run, stimulation can be invoked across each pair of electrodes to determine which pair or set of electrodes is best situated for stimulating the vagus nerve. As described above, the electrodes of a lead can be stimulated to evoke bradycardia, which can be detected by monitoring heart rate. Production of mild bradycardia will confirm that the electrode can stimulate the vagus, and the pair with the lowest threshold will likely be the pair that is chosen for therapy.

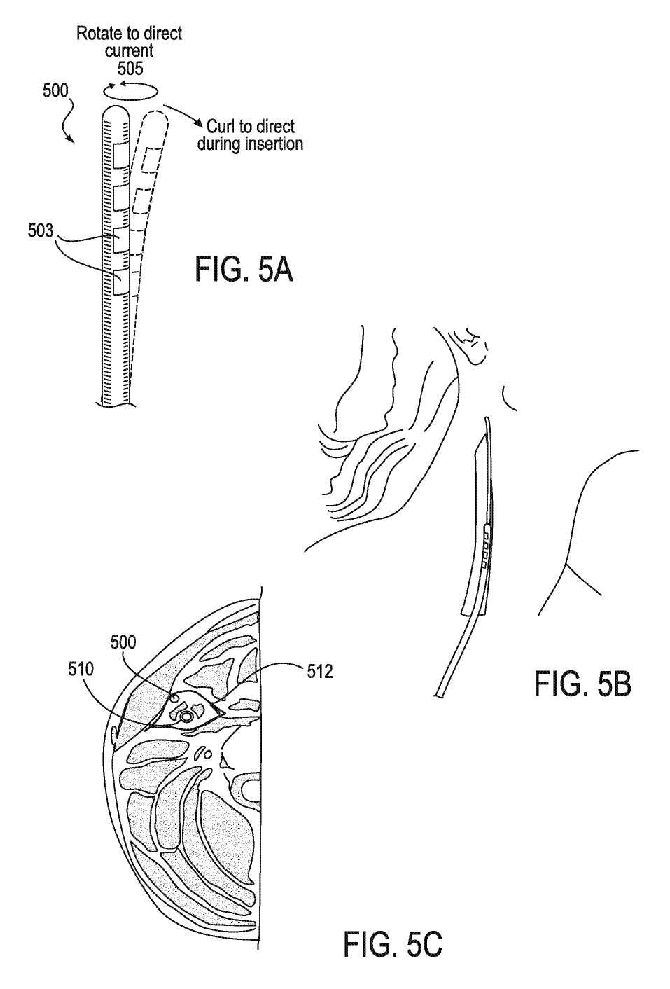

One particular type of system for modulating a subject's inflammatory response (e.g., an intravascular lead system) includes a lead that is configured to be placed within a patient's intra-carotid sheath. For example, an intra-carotid sheath field-effect lead may be used. Intra-carotid sheath field-effect leads (or “sheath FE” leads) are configured to be positioned within a subject's carotid sheath. The carotid sheath typically runs down the neck. The internal jugular vein, vagus nerve, and internal carotid artery extend within the carotid sheath as far as the upper border of the thyroid cartilage. One example of a sheath FE lead is illustrated on FIG. 5A. In this example, the lead 500 includes thin axial electrodes (contacts) 503 that are positioned partially around the body of the lead 500. In some variations these electrodes are rings that encircle the lead body. When the electrodes extend only partially (e.g., ½, ⅓, ¼, etc) around the circumference of the lead body, the lead may be rotated 505 to position the direction of the stimulating field emitted by the lead to stimulate the vagus nerve. Intra-carotid sheath field effect electrodes are one exemplary type of intravascular lead, as illustrated in FIG. 1A, described above.

The lead may be positioned in the carotid sheath in any appropriate manner. For example, the lead may be positioned by a subclavicular approach (using ultrasound guidance). In some variations the lead is introduced percutaneously into the sheath using an introducer. Damage to the surrounding tissue, including the vagus nerve, may be minimized because the electrode body is flexible (and may be “soft”). During positioning of the lead, confirmation of electrical communication with the vagus nerve (and “tuning” of the lead and stimulator and/or controller) may be performed by measuring heart rate. Stimulation of the vagus nerve may cause brachycardia, effect heart rate, allowing testing and optimization of the position of the lead within the carotid sheath by monitoring heart rate. The lead may be positioned so that it does not directly contact the vagus nerve, but is positioned so that electrical stimulation by the lead electrodes will stimulate the vagus nerve and trigger the cholinergic anti-inflammatory pathway (i.e., the inflammatory reflex). The lead may be secured (after positioning) by one or more anchors or retainers. For example, the lead may include axial anchoring regions that expand to engage the tissue. In some variations the lead may be retained by a suture collar. The lead may include a region allowing tissue in-growth (e.g., fibrotic integration), further enhancing anchoring or securing the lead in place.

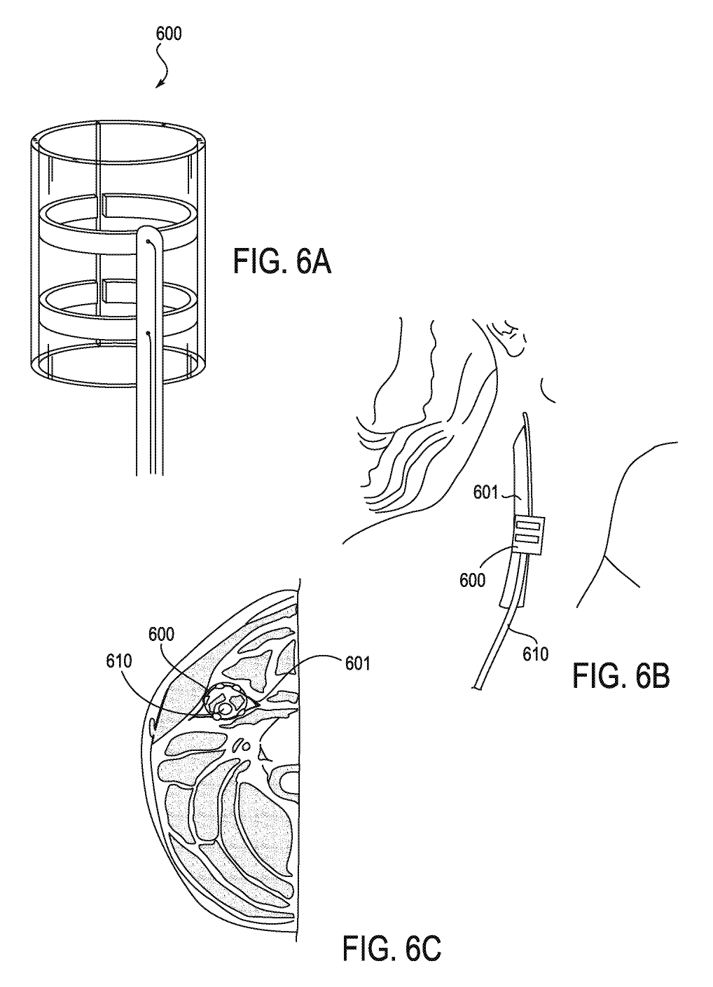

In some variations of the leads described herein, the lead is not inserted into a vessel adjacent to the vagus nerve, but is instead inserted into other (e.g., non-vessel) body regions, such as the region around or immediately within the carotid sheath. For example, FIGS. 6A-6C illustrate a carotid sheath cuff lead (or “sheath cuff” lead). In general a sheath cuff electrode may include an insulator around the sheath with various contact (electrodes) arranged inside the sheath for stimulating the nerve. In this embodiment the lead is configured to be positioned around or over and circumferentially attached to the carotid sheath. The current applied to the lead travels from the electrodes and through the carotid sheath (which includes the vagus nerve, the carotid and the internal jugular vein); surrounding adjacent structures are insulated by the body of the lead.

Similar to the intravascular leads described above, the carotid sheath cuff lead is also an example of a non-contact lead that does not directly contact (and therefore may not contact desensitize) the vagus nerve. The lead may be retained by a suture cuff or other anchor. FIG. 6A illustrates one example of a carotid sheath electrode 600, which is illustrated implanted around the carotid sheath 612 adjacent to the vagus nerve 610 in FIGS. 6B and 6C.

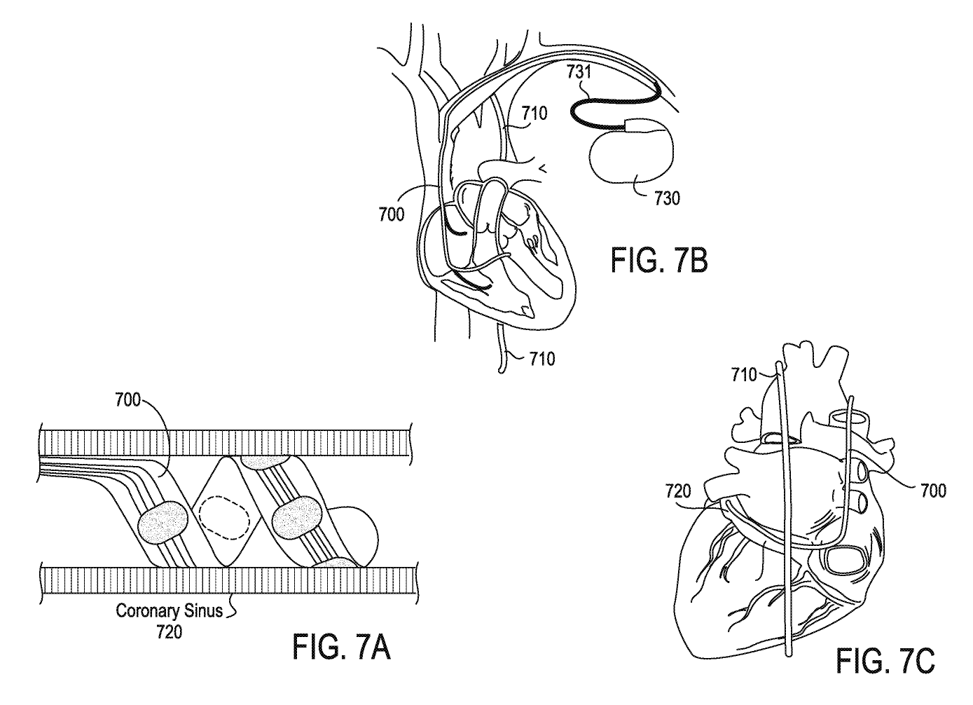

Another example of an intravascular lead is illustrated in FIGS. 7A-7C, which illustrate an intracardiac lead 700. The intracardiac lead 700 shown in FIG. 7A is configured to be inserted into a coronary sinus 720 adjacent to the vagus nerve 710. Intracardiac leads may stimulate the vagus nerve where the vagus nerve 710 passes the heart. For example, an intracardiac lead may be placed in the coronary sinus with outward-facing multipolar leads that may be used to stimulate the vagus nerve. Alternatively, the lead may be placed in the pulmonary artery. FIGS. 8A-8C illustrate an example of an intracardiac inserted into a coronary sinus.

An intracardiac lead, like any of the intravascular leads described herein, may be used to stimulate the inflammatory reflex (e.g., the CAP) through the vagus nerve, and may require only minimal surgery either for acute cannulation or chronic implantation. The vagus nerve runs alongside the heart, e.g., adjacent the pulmonary artery and the coronary sinus. Since these regions may be large enough to accommodate an electrode, and since the tissue is relatively conductive (e.g., ρ=200 Ωcm), the vagus may be stimulated using an intracardiac lead. Placement and anchoring may be confirmed using any of the techniques described above, including stimulation of the various lead electrodes in order to induce bradycardia.

In contrast to the non-contact variations described above, FIGS. 8A-8C illustrate one variation of a sheath electrode 800 that directly surrounds (and may contact) the vagus nerve. As mentioned, contacting the nerve may result in desensitization of the CAP response of the vagus nerve 810. Such vagus nerve cuff leads (or “vagus cuff” leads) typically directly connect to the vagus nerve. These nerve cuffs may be retained via a suture cuff. The outer surface (facing away from the vagus nerve) is typically insulated, preventing current from flowing anywhere except to the surrounded vagus nerve. The contacts on the inside of the cuff may therefore stimulate the vagus nerve.

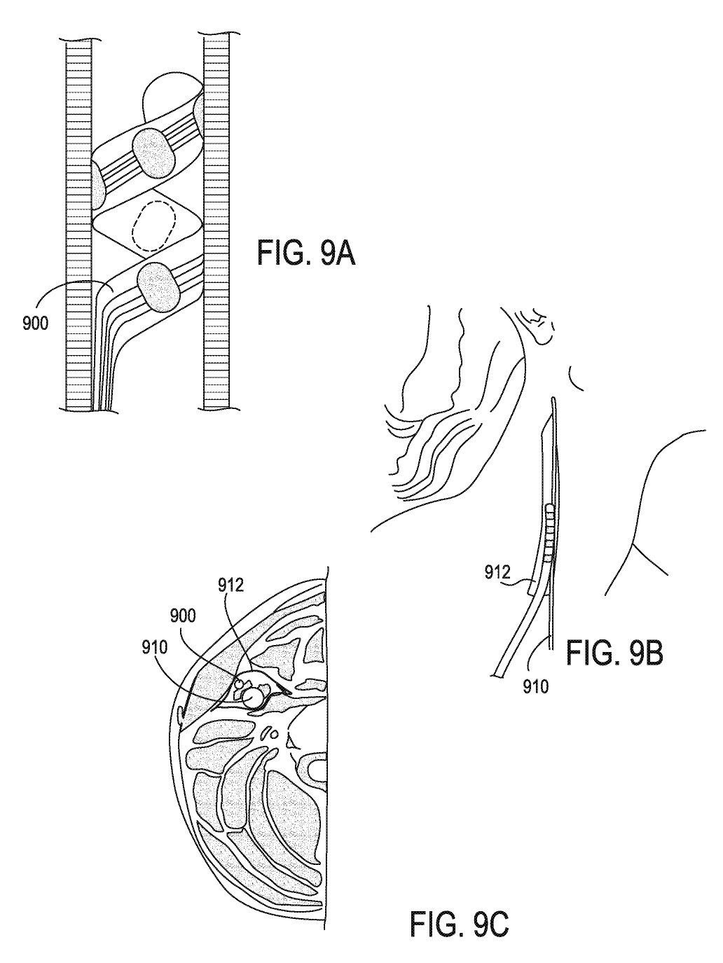

FIGS. 9A-9C show another variation of an intravenous electrode lead, similar to the device shown in FIG. 1A. FIGS. 9B and 9C illustrate the lead of FIG. 9A implanted in a subject's internal jugular vein.

In any of the variations described herein, the lead may be connected directly or wirelessly to other components of the stimulator, including a controller and/or a power source (or diver). For example, FIG. 7B illustrates the intracardiac lead connected via an implantable cable 731 to an implanted housing 730 that holds the controller and a power source (e.g., a pulse generator). The housing may be implanted at the same time that the lead is implanted, and can be positioned away from the vagus nerve. The implantation site may be sub-dermal, but may allow access or communication. For example, the implantation site may be on the left (or right) side of the chest, just below the collar bone (e.g., subclavicular).

Any of the electrodes described herein may be inserted for use as part of a system and/or method for modulation of the CAP (and thereby modulation of the inflammatory reflex).

The examples and illustrations included herein show, by way of illustration and not of limitation, specific embodiments in which the subject matter may be practiced. Other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of modulating inflammation by stimulation of the cholinergic anti-inflammatory pathway, the method comprising:
positioning a flexible lead within a blood vessel;
confirming that each of a plurality of electrodes on the flexible lead are secured against the wall of the blood vessel;
anchoring the flexible lead within the blood vessel; and
modulating the cholinergic anti-inflammatory pathway by applying energy to one or more stimulation electrodes on the flexible lead to stimulate the vagus nerve.

2. The method of claim 1, wherein the step of positioning the lead within the blood vessel comprises positioning the lead within the internal jugular vein.

3. The method of claim 2, wherein the step of positioning the flexible lead in the carotid sheath comprises inserting the lead using a subclavian approach.

4. The method of claim 1, wherein the step of positioning the lead within the blood vessel comprises positioning the lead within the coronary sinus or the pulmonary artery.

5. The method of claim 1, further comprising determining the stimulation electrodes by applying energy from the lead to alter the subject's heart rate.

6. The method of claim 1, wherein the flexible lead comprises a helical flexible lead.

7. The method of claim 1, wherein the step of confirming that each of the plurality of electrodes on the flexible lead are secured against the wall of the blood vessel comprises testing the impedance of each electrode.

8. The method of claim 1, further comprising removing the lead from the blood vessel.

9. The method of claim 1, further comprising implanting a controller, wherein the controller is configured to control the application of energy from the stimulation electrodes.

10. A method of modulating inflammation by stimulation of the cholinergic anti-inflammatory pathway, the method comprising:
positioning a flexible lead in a carotid sheath so that the lead does not contact the vagus nerve;
anchoring the lead within the carotid sheath; and
modulating the cholinergic anti-inflammatory pathway by applying energy to one or more stimulation electrodes on the flexible lead to stimulate the vagus nerve.

11. The method of claim 10, further comprising determining the stimulation electrode or electrodes from among a plurality of electrodes on the flexible lead by applying energy from among the electrodes on the flexible lead and monitoring heart rate.

12. The method of claim 10, wherein the step of positioning the flexible lead in the carotid sheath comprises inserting the lead using a subclavian approach.

13. The method of claim 10, further comprising coupling the flexible lead to a stylet to aid insertion before inserting the flexible lead into the carotid sheath.

14. The method of claim 13, further comprising removing the stylet from the flexible lead after insertion.

15. A method of modulating inflammation by stimulation of the cholinergic anti-inflammatory pathway, the method comprising:
positioning a carotid sheath cuff lead around a carotid sheath so that the carotid sheath cuff lead does not contact the vagus nerve;
anchoring the carotid sheath cuff lead around the carotid sheath so that stimulation electrodes located within the carotid sheath cuff lead are oriented towards the vagus nerve within the carotid sheath; and
modulating the cholinergic anti-inflammatory pathway by applying energy to one or more stimulation electrodes within the carotid sheath cuff lead to stimulate the vagus nerve.

16. The method of claim 15, wherein the step of positioning the carotid sheath cuff lead around the carotid sheath comprises surgically cutting down to the carotid sheath.

17. The method of claim 15, further comprising suturing the carotid sheath cuff around the carotid sheath.

18. The method of claim 15, wherein the carotid sheath cuff is insulated on the outer surface of the cuff to prevent stimulation of surrounding tissue.
